(19) United States
(12) Patent Application Publication
(10) Pub. No.: US 2013/0109008 A1
(43) Pub. Date: May 2, 2013

(54) METHOD AND APPARATUS FOR GROWING PHOTOSYNTHETIC ORGANISMS

(71) Applicants: Marc Rene Stammbach, Killara (AU); Rocky De Nys, Townsville (AU); Kirsten Heimann, Townsville (AU); Arnold Mangott, Townsville (AU); Howard Kneebone, Goulburn (AU)

(72) Inventors: Marc Rene Stammbach, Killara (AU); Rocky De Nys, Townsville (AU); Kirsten Heimann, Townsville (AU); Arnold Mangott, Townsville (AU); Howard Kneebone, Goulburn (AU)

(73) Assignee: MBD ENERGY LIMITED, East Melbourne, Victoria (AU)

(21) Appl. No.: 13/807,037

(22) PCT Filed: Jul. 1, 2011
(86) PCT No.: PCT/AU2011/000829

§ 371 Date: Dec. 27, 2012

(30) Foreign Application Priority Data
Jul. 1, 2010 (AU) 2010902933
Feb. 21, 2011 (AU) 2011900589

(51) Int. Cl. C12M 1/36

(52) U.S. Cl. CPC C12M 1/36
USPC 435/3; 435/286.6

(57) ABSTRACT

A biological cultivation system for the culture of photosynthetic organisms including at least one cultivation chamber permitting exposure of the culture medium to natural and/or artificial light and including; a light transmissive wall or walls defining a gas space; and a culture medium containment area below the gas space; one or more fluid inlets positioned within the culture medium containment area; and one or more gas outlets in communication with the gas space; a control unit operatively connected to a gas flow control device, the gas flow control device controlling the flow of gas in through the fluid inlets and out through the fluid outlets to control the conditions within the cultivation chamber.

### FIELD OF THE INVENTION

The present invention relates to cultivation chambers and methods for the production of photosynthetic organisms, in particular algae and including microalgae and macro algae.

### BACKGROUND OF THE INVENTION

The culturing of photosynthetic organisms, particularly microalgae and cyanobacteria, has become the focus of much interest due to the multiple applications for such microorganisms. Firstly, the culturing of photosynthetic microorganisms can utilise waste carbon dioxide (CO₂) and nutrients (for example from sewerage or agriculture outputs) and, in the presence of light, convert these into biomass. Secondly, the produced biomass has the potential for a multitude of uses including: the extraction of oils, which may then be converted into biodiesel; as raw materials for the bioplastics industry; to extract nutraceutical, pharmaceutical and cosmetic products; for animal feed and as feedstock for jetfuel, pyrolysis and gasification plants.

The present invention aims to address one or more of the difficulties of the systems known in the art for culturing photosynthetic organisms, particularly algae.

### SUMMARY OF THE INVENTION

In a first aspect, the present invention provides a biological cultivation system for the culture of photosynthetic organisms including
at least one cultivation chamber permitting exposure of the culture medium to natural and/or artificial light and including;
   a light transmissive wall or walls defining
   a gas space; and
   a culture medium containment area below the gas space;
   one or more fluid inlets positioned within the culture medium containment area; and
   one or more gas outlets in communication with the gas space;
a control unit operatively connected to a gas flow control device, the gas flow control device controlling the flow of gas in through the fluid inlets and out through the fluid outlets to control the conditions within the cultivation chamber.

In an embodiment, the biological cultivation system may further include a plurality of cultivation chambers. This permits both flexibility of production and centralisation of system control. The cultivation chambers may be interconnected, e.g. via a manifold means, in series or in parallel. Preferably 10 to 200 cultivation chambers may be joined; more preferably 20 to 60. The cultivation chamber array may include use with parallel like-configured vessels or a combination of 2 or more dissimilar growth medium vessels. This may include, but is not limited to, multiple cultivation chambers in parallel or a combination of bags and open raceways.

Thus, in an alternative embodiment of the present invention, the biological cultivation system may further include a plurality of cultivation chambers wherein the cultivation chambers include
one or more chambers formed of a flexible material; and
one or more chambers including a pair of opposed substantially rigid walls enclosed by a light transmissible section.

The rigid cultivation chamber(s) may permit the cultured organisms to be rested in comparatively low light where required. The rigid cultivation chamber may also include the control unit as described above and a balance tank.

In a further aspect of the invention, there is provided a method of cultivating photosynthetic organisms may include:
(a) providing a cultivation chamber including:
   (i) at least one light transmissive wall or walls defining:
      a gas space; and
      a culture medium containment area below the gas space;
   (ii) one or more gas inlets and one or more gas outlets in communication with the gas space;
   (iii) one or more fluid outlets and outlets communicating with the culture containment are; and
   (iii) a gas flow control means;
(b) introducing into the cultivation chamber a culture medium and an inoculate of photosynthetic organisms, the cultivation chamber permitting exposure of the culture medium to a natural and/or artificial light;
(c) controlling the flow of gas in through the gas inlets and out through the gas outlets using the gas flow control device, wherein the flow of gas drives evaporation from the culture medium and/or controls the temperature of the cultivation chamber; and
(d) allowing the photosynthetic organisms to grow in the presence of light.

In one embodiment, the cultivation chamber includes one or more fluid ports to allow for the introduction and removal of the culture medium. Preferably, the one or more fluid ports include a regulator to control the introduction and removal of the culture medium from the cultivation chamber. More preferably, the regulator is a valve, preferably a ball valve.

Photosynthetic organisms convert carbon dioxide, water and nutrients into biomass in the presence of light. Therefore, the growth of these photosynthetic organisms enables carbon dioxide emitted as, for example, flue gas from a power plant, refinery or cement kiln, liquid natural gas production or coal seam gas, to be recycled as biomass rather than being released into the atmosphere. The conditions controlled within the cultivation chamber include the pH and CO₂ content of the culture medium, the evaporation rate and temperature in the cultivation chamber. Adding gases rich in CO₂ into an algal slurry decreases the pH of the solution/slurry. As CO₂ is consumed the pH will increase. Therefore by balancing the rate of supply of CO₂ and consumption of CO₂, a stable and optimal pH can be maintained which ensures adequate carbon concentrations are available for photosynthesis to take place and new bio-material to be formed as cells grow and multiply.

The fluid inlets for the culture medium containment area are positioned along a base portion of the culture medium containment area.

The photosynthetic organisms are selected from the group consisting of macroalgae, microalgae and cyanobacteria. Preferably the photosynthetic organisms are microalgae.

The concentration of CO₂ introduced may be varied by varying the amount of air mixed with the CO₂. For example, CO₂-containing flue gas may be diluted with air, depending on the CO₂ requirements of the photosynthetic organisms. During periods of darkness, for example at night when natural light is used, the amount of CO₂ may be decreased while maintaining a constant gas flow by increasing the amount of air in the mixture. The air to be mixed with the CO₂ source may be filtered to remove certain particulate matter, for example using a particulate air filter, more preferably a high efficiency particulate air (HEPA) filter.

The culture medium may be any suitable medium for the growth of the desired photosynthetic organisms. The culture medium may be based on fresh or saline water and may include waste water from industrial processes or sewerage treatment systems. The culture medium may include additional nutrients including iron sulphate and the like.

The photosynthetic organisms may be selected from any suitable organisms and may be cultured as a single species in monoculture or two or more species in the same cultivation chamber. A polyculture is preferred as this may provide resilience to changes in the environment due to e.g. temperature, make-up of nutrients and salinity Photosynthetic organisms that produce useful ingredients for the chemical, biodiesel, pharmaceutical or nutraceutical industries are preferred. Suitable photosynthetic microorganisms include cyanobacteria (blue-green algae) and algae, preferably microalgae or macroalgae. The microorganisms may grow in fresh or salt water. Examples of photosynthetic microorganisms that may produce useful ingredients/feedstocks include, but are not limited to, those belonging to the following genera: Chiamydomoas; Chaetoceros; Dunaliella; Haematococcus; Isochrysis; Nannochloropsis; Porphyridum; Picochlorum (synonym Nannochloris); Pleurochrysis; Rhodomoas, Spriulina.

A preferred microalgal strain may be a fast growing strain. The strain may exhibit high lipid content and be salinity tolerant. A Nannochloropsis strain or mixture of strains is particularly preferred. The method of the present invention may also be utilised to culture macroalgae, for example those of the genera Ulva, Cladophora; Chaetomorpha. or Oedoginium.

The photosynthetic organisms produced according to the present invention have a number of potential uses. Oil (e.g. triglycerides) may be extracted from the microorganism and this oil may be used for: biodiesel production (e.g., using known transesterification processes); as a raw material for the production of plastics and for the synthesis of jet and other fuels. The cake component of the biomass that is left after the extraction of oil may be used as: feed for the livestock industry; fertilizer production; biomass for bio-plastic production or biomass for energy and jet fuel production and/or pyrolysis. Photosynthetic organisms may also produce other useful products, such as nutraceuticals (e.g. omega 3 and 6 fatty acids; antioxidants, such as astaxanthin and pigments, such as β-carotene), phycocolloids, triglycerides and other ingredients for the pharmaceutical and cosmetics industries.

In a preferred embodiment, fluid inlets are positioned along a base portion of the culture medium containment area. The first and/or second gas may be an oxygen-containing gas, e.g. air. The second gas may be the same as, or different to the first gas. The first gases may include carbon dioxide (CO₂). Where CO₂ is included, the gas may function to provide carbon to the photosynthetic system and/or reduce the pH of the circulating fluid, e.g. water.

In one embodiment, one or more walls of the cultivation chamber is(are) composed of a flexible material, which may allow for the inflation of the cultivation chamber. Within the context of the invention, a flexible material is a pliable material which does not have a rigid shape. Such a flexible material includes, but is not limited to, a plastic-type film. In a preferred embodiment, the cultivation chamber is in the form of an enclosed flexible plastic structure of tube-like configuration, such as a plastic bag-type structure. In a preferred embodiment, one or more walls of the cultivation chamber are light-transmissible and the walls may be integrally formed in a tubular shape. The cultivation chamber is preferably horizontally oriented producing a flat base. The base preferably has a slope of 1-5° towards the discharge end of the chamber. This assists the flow of cultured algal slurry towards the discharge outlet.

The plastic-type film may be selected to permit transmission of light at a pre-determined wavelength. A polyethylene film, e.g. a linear low density polyethylene film and/or medium density film may be used. The plastic-type film may include pigments e,g. mineral oxides, such as titanium and/or ferric oxides, and/or other light controlling additives to permit control of light transmission. In one embodiment a material permitting UV light transmission of approximately 20% to 65%, preferably 25% to 60%, may be used.

In a more preferred embodiment, the cultivation chamber is inflatable. Where the cultivation chamber is inflatable, the inflation of the cultivation chamber may be maintained by the flow of gas achieved through the introduction of gas into the chamber through the gas inlets and out through the gas outlets. The gas may be introduced into the cultivation chamber through one or more gas inlets positioned above and/or below the surface of the culture medium.

In a further preferred embodiment, the cultivation chamber may include
a pair of opposed substantially rigid side walls; and
a light-transmissible section connecting and providing a canope enclosing the side walls.

The use of substantially rigid side walls may improve the strength and longevity of the cultivation chamber(s). The side walls may include a pair of opposed bulkheads, e.g. steel bulkheads.

Thus, in an alternative embodiment of the present invention, the biological cultivation system may include a plurality of cultivation chambers wherein the cultivation chambers include
one or more chambers formed of a flexible material; and
one or more chambers including a pair of opposed substantially rigid walls enclosed by a light transmissible section.

Whilst the direct control of light transmissibility in the light-transmissible sections of the cultivation chamber may limit photoinhibition of the growth of the photosynthetic organisms, additional light control may be required during particular seasons and/or particular times of the day. Accordingly, the cultivation chamber may further include a secondary light control device. The light control device may be of any suitable type. The secondary light control device may be fixed and/or variable. A shade or filter, such as a shade sail or cloth may be provided.

Light level shading may be fixed due to pre-calculated geographic characteristics of ambient light levels.

Shading may be variable via external light permeable covers such as shade sails due to variable geographical and chronological ambient light conditions. Variable parameters that dictate a variable control of shading would be but are not limited to:
(i) time of day and sunlight intensity
(ii) geographic latitude and/or
(iii) actual light requirement of an individual species of algae strain both macro and micro varieties of algae within a controlled growth process.

Preferably a number of fluid inlets are positioned throughout the cultivation chamber. In a preferred embodiment, the fluid inlets are positioned at the base of the cultivation chamber, preferably along the length of the base of the cultivation chamber.

In a further preferred embodiment, fluid inlets are positioned along conduits at the base of the cultivation chamber, wherein the conduits are adapted to carry and distribute the flow of gas. The gas inlets are preferably positioned along the conduits at intervals that allow for a substantially even distribution of gas flow along the length of an elongated cultivation chamber. The gas outlets may be designed to release excess gas pressure that may build up in the flexible cultivation chamber.

In a preferred embodiment, the gas outlets may include a valve system, preferably a one-way valve system. The use of one-way valves may reduce the risk of contamination of the cultivation chamber from outside air, whilst permitting removal of excess oxygen etc.

In a further embodiment, gas is passed into the cultivation chamber through fluid inlets both above the surface of the culture medium. and below the surface of the culture medium. The introduction of the gas above the surface of the culture medium allows for a modification of the atmosphere in the cultivation chamber.

Cultures of any kind and in any embodiment can become contaminated by rogue (unwanted organisms. Accordingly, in a preferred aspect of the present invention, the method may further include
providing an effective amount of a selective biocide; and
treating the culture medium with the selective biocide to reduce or eliminate growth of unwanted organisms.

The unwanted organism may be a parasite, bacterium, fungal or algal strain. The biocide may accordingly include a pesticide, bactericide, fungicide or algaecide or a combination thereof.

A source of light is required for the organisms to photosynthesise. Any suitable source of light may be used including natural light and artificial light or a combination of natural and artificial light. Artificial light may be provided by any suitable light source. In one embodiment, the artificial light source is provided by light-emitting diodes (LEDs). An artificial light source may be provided to extend the length of time per day that the organisms continue to photosynthesise beyond daylight hours. Accordingly, in one embodiment, the cultivation chambers and methods of the present invention are adapted to alternate between using natural and artificial light as required.

In a further preferred aspect of the present invention, the method of cultivating photosynthetic organisms where the cultivation chamber further includes a gas flow control device; the method further including the step of controlling the flow of gas in through the fluid inlets and out through the fluid outlets using the gas flow control device, wherein the flow of gas drives evaporation from the culture medium and/or controls the temperature of the cultivation chamber. The gas flow control device is preferably a fan. The gas flow control device is preferably situated at one end of an elongate cultivation chamber. The gas flow control device may function to create a positive atmospheric displacement differential between the liquid culture medium and the gas space. This may function to retard the escape of the first gas, e.g. a CO₂-containing gas from the culture medium and increase its residence time in the culture medium. The longer residence time of e.g. CO₂ may assist in efficient dosing of the culture medium to enhance algal growth.

When using an enclosed bioreactor that is not open to the atmosphere, usage of a positive pressure displacement differential between the liquid medium and the enclosed atmospheric space above the liquid interface area may function to retard the escape of CO₂ out of solution of the growth medium liquid. The longer residence time of CO₂ in water solution will assist in more efficient dosing of CO₂ for algae growth and less CO₂ to escape to atmosphere without being taken up by algae growth.

Improved atmospheric pressure control is thus achieved by:
(i) metering of gas delivery volume within the bag/bioreactor on the delivery feed side and
(ii) excess gas pressure being released from air gap above liquid medium via pressure release mechanisms such as either controllable or fixed pressure point relief valves.

It has been found that evaporation from the culture medium may enable a degree of control over the temperature within the cultivation chamber. This assists in the culture of the microorganisms, as temperature control is important to gaining optimal growth and/or optimal production of the relevant chemicals by the microorganisms (such as triglycerides as ingredients for biodiesel).

In a preferred embodiment, the gas flow control device further includes a temperature control. It has been found that a cultivation chamber, e.g. a photo-bioreactor, which is an enclosed unit, may suffer from a so called “greenhouse effect”. The exposure to light may result in an unacceptable increase in temperature within the chamber which may inhibit algal growth or even kill the algae.

The temperature control may function to lower or raise the temperature of the cultivation chamber. The temperature control may include an evaporative cooling or air conditioning system. The temperature control may include a heat exchange system.

To counteract any unwanted salinity increases associated with the evaporation of the water from the culture medium, additional water may be added to the culture medium. This additional water may be in any suitable form, for example as fresh water or aquaculture waste water.

A further mechanism for controlling the temperature of the culture medium using the above aspects of the present invention is to control the rate at which gas is introduced into the cultivation chamber and/or the temperature of the introduced gas. For example, heat loss at lower than optimal ambient temperatures may be reduced by lowering the amount of gas at a temperature lower then ambient temperature being introduced into the culture medium, thereby reducing the mixing of the medium and resultant heat exchange. Accordingly, in darkness the gas flow may be reduced and may be stopped completely, to at least partially maintain the temperature of the cultivation medium during low night time ambient temperatures.

Furthermore, by varying the temperature and composition of the gas introduced into the cultivation chamber the temperature of the liquid culture medium may be varied. For example, if using enriched CO₂ from flue gas as an input, the flue gas may be maintained at a higher temperature to counteract the effect of low ambient temperatures. Accordingly, the flue gas may be introduced at a higher temperature where the temperature of the culture medium needs to be increased. Conversely, where the temperature of the liquid culture medium needs to be reduced, the flue gas may be cooled further before introducing to the cultivation chamber.

Alternatively, increasing the amount of air introduced into the cultivation chamber may aid in the cooling of the cultivation medium. This air may be introduced by bubbling from under the surface of the cultivation medium or by being passed over the surface of the cultivation medium.

Alternatively, the temperature of the cultivation medium may be controlled by circulating it directly or indirectly over a suitable heat exchanger, such a cooling tower or a boiler.

The cultivation chamber may be of any suitable size to cultivate the required amount of the photosynthetic organisms.

In a preferred embodiment, the cultivation chamber may be about 1 metre to about 10 metres, more preferably about 2 metres to about 6 metres in width. In a particularly preferred embodiment, the cultivation chamber of the present invention is about 3 metres in width.

In an alternative embodiment, the cultivation chamber may be about 5 metres to about 250 metres, more preferably about 10 metres to about 100 metres in length. In a particularly preferred embodiment, the cultivation chamber is about 50 metres in length.

In one embodiment, the level of the culture medium in the cultivation chamber is controlled by the regulation of the intake of culture medium through one or more liquid ports, which act as inlets and/or outlets for the passage of liquid into and out of the cultivation chamber. It has been found that improved photosynthetic culture may be achieved by limiting the flow rate of culture medium through the cultivation chambers. This is possible as a consequence of permitting the gas to pass through the culture medium and thus provide adequate mixing thereto,

Accordingly, in a further aspect, the control unit may further include

a culture medium input system; the cultivation chamber being flow-connected to the control unit the liquid inlets and outlets permitting controlled circulation of the culture medium.

The passage of the culture medium through the fluid ports in one or both directions is preferably regulated by one or more valves. The valves may be controlled by the control unit or may be externally controlled valves. The valves may be reactive to the level of the culture medium in the cultivation chamber, thereby allowing for the emptying (for example, for harvesting the photosynthetic organisms) and refilling of the cultivation chamber. Alternatively or in addition, the valves may be reactive to the concentration of algae generated in the growth column array. In a preferred embodiment the valves are ball valves.

In a further embodiment, the level of the culture medium in the cultivation chamber is measured by means of one or more sensors.

The control unit may further include
a balance tank for maintaining fluid flow; and optionally
a sampling unit to permit testing of the culture medium.

The sampling unit may further provide an input feed device. The input feed device may provide a single location for nutrient addition and/or other dosing tasks. In a preferred embodiment where a source of a selective biocide is provided, this biocide may be added via the input feed device.

Usage of a single location within an identified process of algae growth and harvesting to perform input feed or dosing tasks and process control function for a parallel grouped array of multiple ponds, growth column arrays, photobioreactors (PBR's), cultivation chambers, open raceways and other algae growth medium water retention vessels may significantly simplify unit design and function.

In a particularly preferred embodiment, control unit may include drive controller(s) in combination with a programmable logic control (PLC). In this embodiment, the drive controller may function to control both culture growth and pump control.
(a) The control unit may include a vector based combination drive and input/output (I/O) interface to combine pump and process control function.
(b) The combination drive and process controller has the capability to be presented/programmed in either conventional PLC (programmable logic control-ladder configuration or function block based presentation) or background depicted decompiled firmware programming to an integrated circuit chip that cannot be edited beyond normal MMI/HMI presented operator changeable parameters.
(c) Programming languages specifically intended are; (Function block diagram), LD (Ladder diagram), ST (Structured text), IL (Instruction list, similar to assembly language) and SFC (Sequential function chart), and modular programming.
(d) The VSD controller may include a DSP (digital signal processor) that can operate concurrently as a PLC CPU (central processor unit) for required algae growth process control and as a variable speed pump controller.
(e) A single master DSP may also control multiple slave pump controller and growth controller units.

The control unit may be designed to function on AC and/or DC power input. The control unit may accordingly further provide for an AC input to DC bus, e.g. via a rectifier of an inverter to AC output.

The control unit may further include a drive controller(s) to control the pumping rates of the culture medium. Preferably the drive controller(s) are variable speed drive controllers (VSP). Variable speed drives to control pumps both AC and DC powered allow variable pumping rates of algae growth medium for use in process control. The pumping rates of water may be varied upon growth requirements or harvesting/make up Input/output (I/O) Variable Frequency Drives (VFD), Pulse width modulated (PWM) and/or Vector type pumping drive controllers. DC drive controllers can be either variable voltage control or with pulse width modulation.

It has been found that it is preferable to ‘starve’ the organisms of nutrients for a period, e.g. of 1 to 5 hours, prior to harvesting to increase overall lipid content achieved.

Accordingly, the method of the invention further includes the steps of reducing or
eliminating the nutrient content of the culture medium for a pre-determined period; and
harvesting the photosynthetic organisms.

The biological cultivation system for the culture of photosynthetic organisms may further include (i) at least one vertically oriented growth column including
a light transmissible conduit;
one of more fluid inlets in communication with the conduit; and
one or more fluid outlets; and
wherein a fluid outlet of the growth column is flow-connected to a fluid inlet of the cultivation chamber.

It has been found that by utilising the vertical growth column, the growth efficiency of the biological cultivation system may be significantly improved.

According to a further aspect, there is provided a biological cultivation system for the culture of photosynthetic organisms including:

(i) a vertically oriented growth column including
a light transmissible conduit;
one of more fluid inlets in communication with the conduit; and
one or more fluid outlets; and
   (ii) a cultivation chamber permitting exposure of the culture medium to natural and/or artificial light and including;
a light transmissive wall or walls defining
   a gas space; and
   a culture medium containment area below the gas space;
one or more fluid inlets positioned within the culture medium containment area; and
one or more gas outlets in communication with the gas space;
wherein a fluid outlet of the growth column is flow-connected to a fluid inlet of the cultivation chamber.

It has been found that by utilising the vertical growth column, the growth efficiency of the biological cultivation system may be significantly improved.

In a preferred form of the invention, the light transmissive conduit includes a light transmissive inner conduit and a light transmissive outer conduit surrounding and in fluid communication with the inner conduit. In order to establish a flow circulation system, it is preferable for the fluid inlet or inlets for the vertical growth column to be provided to either of the inner or outer conduits and the fluid outlets provided to the other of the inner or outer conduit. In this way, biomass, media and gas passes up either the inner or outer conduit and the biomass and media then descends down the other of the inner or outer conduit. In circumstances where the biomass density is increased during the up-flow to affect the intensity of light reaching the biomass, preferably the biomass and media travel up the outer conduit and down the inner conduit.

Suitable gases and/or liquid nutrients may be introduced into the vertical growth column and cultivation chamber of the present invention to aid the growth of the photosynthetic organisms. Such gases or liquids may be selected from carbon dioxide (CO₂); fertilisers and waste from aquaculture and agriculture (for example: trout, salmon, cattle, pig and chicken farms). The CO₂ may be from any suitable source and may be from air or in a concentrated form. Examples of suitable concentrated sources of CO₂ include, but are not limited to, flue gases, kiln and incineration gases and gases from anaerobic digestion. In a preferred embodiment, the source of CO₂ is a flue gas, more preferably desulphured flue gas (DFG).

In the preferred embodiment in which a growth column or columns is/are included in the biological cultivation system, the culture medium may include a concentrated slurry containing a mixture of culture medium and photosynthetic organisms (e.g. an algal slurry). The culture medium may include selected nutrients and/or trace elements to enhance growth. For example a culture medium including iron sulphate has been found to enhance algal growth.

The method of the invention may further include the steps of
(a) providing
   (i) a vertically oriented growth column including a light transmissive conduit; and
(b) introducing into the vertical growth column, a culture medium and an inoculate of photosynthetic organisms;
(c) introducing a first gas into the vertical growth column; and allowing the photosynthetic organisms to grow in the presence of light;
(d) introducing the product of step (c) into the cultivation chamber, the cultivation chamber permitting exposure to natural and/or artificial light;
(e) introducing a first gas through the inlet(s) within the containment area, wherein the flow of gas thereby mixes the culture medium;
(f) introducing a second gas through the gas inlet(s) into the gas space, wherein the second gas functions to control the temperature of the gas space; and
(g) allowing the photosynthetic organisms to grow further in the presence of light.

According to a further aspect, there is providing a method of cultivating photosynthetic organisms including the steps of:
(a) providing
   (i) a vertically oriented growth column including a light transmissive conduit; and
   (ii) a cultivation chamber including a wall or walls defining a gas space and a culture medium containment area below the gas space;
(b) introducing into the vertical growth column, a culture medium and an inoculate of photosynthetic organisms;
(c) introducing a first gas into the vertical growth column; and allowing the photosynthetic organisms to grow in the presence of light;
(d) introducing the product of step (c) into the cultivation chamber, the cultivation chamber permitting exposure to natural and/or artificial light;
(e) introducing a first gas through the inlet(s) within the containment area, wherein the flow of gas thereby mixes the culture medium;
(f) introducing a second gas through the gas inlet(s) into the gas space, wherein the second gas functions to control the temperature of the gas space; and
(g) allowing the photosynthetic organisms to grow further in the presence of light.

The biological cultivation system as described above includes a vertically oriented growth column which may be substantially filled with a culture medium inoculated with a selected photosynthetic organism or mixture of organisms. The vertically oriented growth column may include an inner and an outer conduit.

Gas may be passed into the base of the inner conduit via the gas inlets and may be bubbled into the culture medium. The introduction of gas allows for the mixing of the culture medium and assists in the distribution of the gases, nutrients, light and heat throughout the culture medium. In a preferred embodiment, a first gas is introduced in a substantially continuous manner while the photosynthetic organisms are photosynthesising (in the presence of light). The gas flow also permits movement of culture medium from the inner conduit to the outer conduit. This improves the exposure of the organisms to light as the concentration of organisms increases during growth.

The first and/or second gas may be an oxygen-containing gas, e.g. air. The second gas may be the same as, or different to the first gas. The first gas may include carbon dioxide (CO₂). Where CO₂ is included, the gas may function to provide carbon to the photosynthetic system and/or reduce the pH of the circulating fluid, e.g. water.

In a preferred embodiment of the present invention, an array of vertical growth columns may be used. The vertical growth columns may be the same or different. One or more vertical growth columns may include a light transmissible inner conduit and a light transmissible outer conduit, and/or one or more growth columns may include a single light transmissible column.

The vertical growth columns may be arranged in any suitable manner. The columns may be arranged in series or in parallel. When in series, the algal slurry from one column becomes the feedstock for an adjacent column. The concentration within the slurry may thus increase throughout the array.

Once a selected concentration of e.g. algae is achieved, the algal slurry may be passed through the fluid outlet(s) to the cultivation chamber. Typically the growth columns can sustain growth of algae between 30-75 grams dry weight (DW) per square meter compared to 20-35 grams DW per square meter for the cultivation chambers.

Gas may then be passed into the cultivation chamber via the fluid inlets. Where the fluid inlets are situated below the surface of the culture medium containing the photosynthetic organisms, gas may be bubbled into the culture medium. The introduction of gas below the surface of the culture medium allows for the mixing of the culture medium and assists in the distribution of the gases, nutrients, light and heat throughout the culture medium. In a preferred embodiment, a first gas is introduced in a substantially continuous manner while the photosynthetic organisms are photosynthesising (in the presence of light).

Accordingly, in a further aspect the present invention provides a product extracted from photosynthetic organisms produced in accordance with the method of the present invention. In one embodiment, the product is selected from the group consisting of an oil; glycerol; omega 3 and 6 fatty acids; astaxanthin; and 6-carotene. In another embodiment, the product is biomass cake, such as algae cake.

Where the cultivation chamber is inflatable, gas outlets may be provided above the level of the cultivation medium to release the pressure that is built up through the gas which has been bubbled through the cultivation medium, for example from the base of the cultivation chamber.

Accordingly, in a further aspect the present invention provides a method for the conversion of carbon dioxide to algal biomass including the steps of:
cultivating algal photosynthetic organisms by the method described above in the presence of light wherein the second gas is carbon dioxide.

In a further aspect, the present invention provides a method of recycling emitted carbon dioxide by utilising the emitted carbon dioxide as an input in the production of photosynthetic organisms using the method of the present invention. The emitted carbon dioxide may be flue gas, kiln gas, incineration gas and gas from anaerobic digestion.

Where the CO₂ is provided from flue gas, the flue gas is preferably cooled and partly scrubbed of pollutants such as SOx, dust, heavy metals etc before it is introduced into the cultivation chamber. Heavy metals, SOx and dust remaining in the flue gas after partial scrubbing may provide micronutrients required for the growth of the photosynthetic organism. Such micronutrients may then be added to the culture medium, either directly or with additional treatment, e.g. the selective removal of heavy metals.

In a further aspect of the present invention there is provided a method for the separation of concentrated microorganism biomass from a slurry of biomass in an aqueous media into fractions including the steps of:
(1) providing concentrated microorganism biomass having substantially intact cells;
(2) homogenising the biomass using a mechanical homogeniser to disrupt the cells of the microorganisms; and
(3) separating the homogenised biomass into fractions.

In a further aspect of the invention, there is provided a photosynthetic growth system including a plurality of cultivation chambers arranged in two or more sections, wherein the sections are connected in series and the cultivation chambers in each section is of a greater volume/capacity than the cultivation chambers of the previous section in the series or the total volume/capacity of cultivation chambers in each section is greater than the previous section.

In a preferred embodiment of this aspect, the cultivation chambers in the first section may be vertical growth columns as described above and the subsequent sections may include any of the cultivation chambers described earlier. In each section, the cultivation chambers may be connected in parallel or in series. A further aspect of the invention may include a method of producing a photosynthetic organisms in the system including a plurality of sections as described above.

### DETAILED DESCRIPTION OF THE DRAWINGS

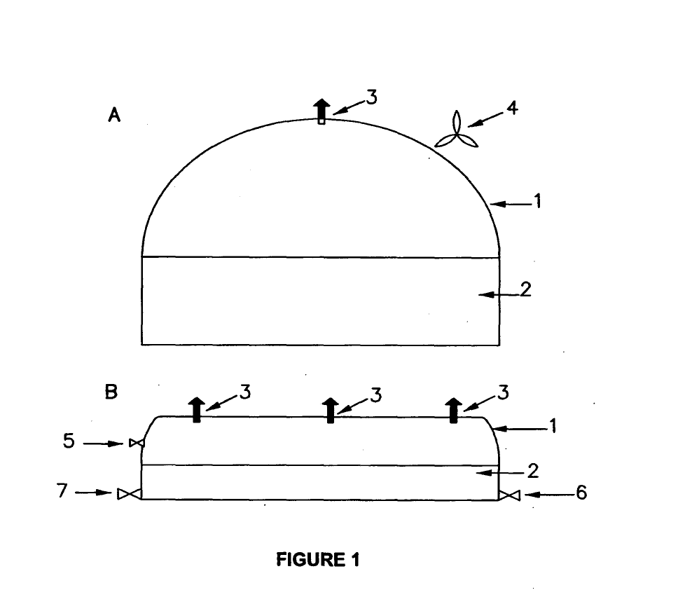

FIG. 1(A) is a front view bag cultivation chamber according to a first embodiment and

FIG. 1(B) is a side view of the embodiment of FIG. 1(A)

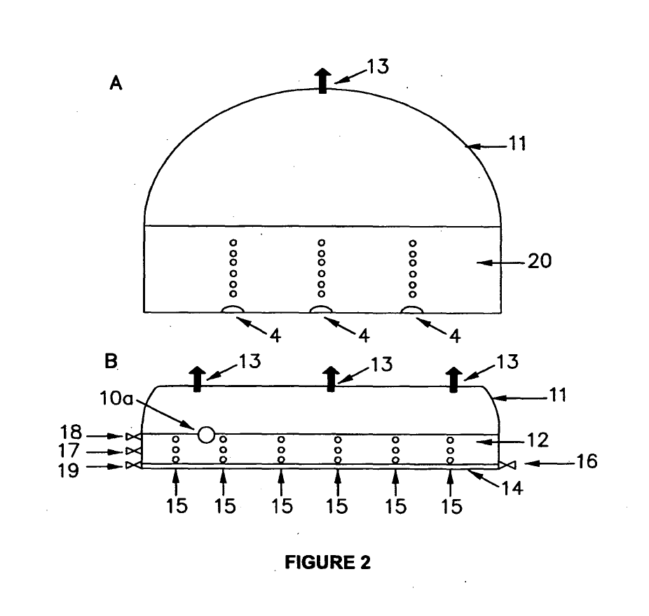

FIG. 2(A) is a front view of a second embodiment of the bag cultivation chamber and

FIG. 2(B) is a side view of the embodiment of FIG. 2(A)

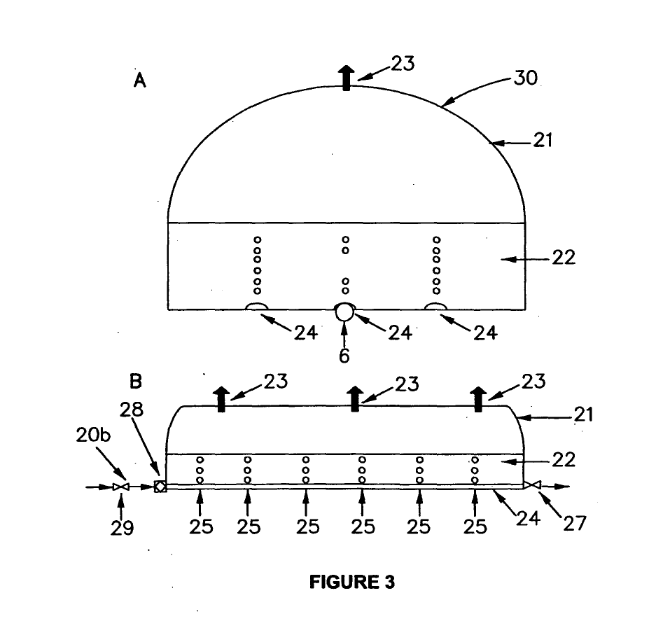

FIG. 3(A) is a front view of a third embodiment of a bag cultivation chamber and FIG. 3(B) is a side view of the embodiment of FIG. 3(A)

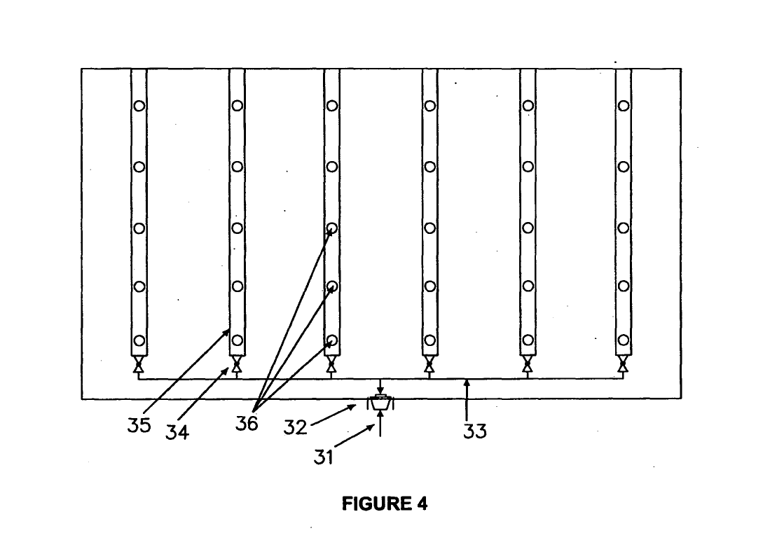

FIG. 4 is a top view the embodiment of FIG. 3(A)

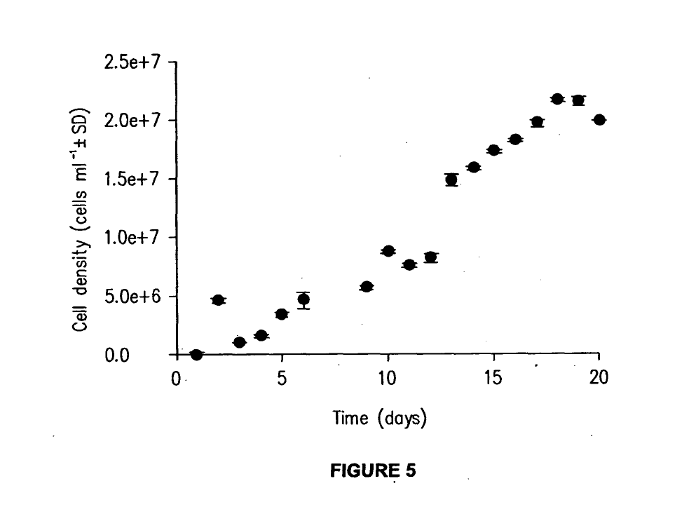

FIG. 5 is a graph of Cell density (cells mL-1) from day 1 (inoculation) to day 20. Average±standard deviation, n=3.

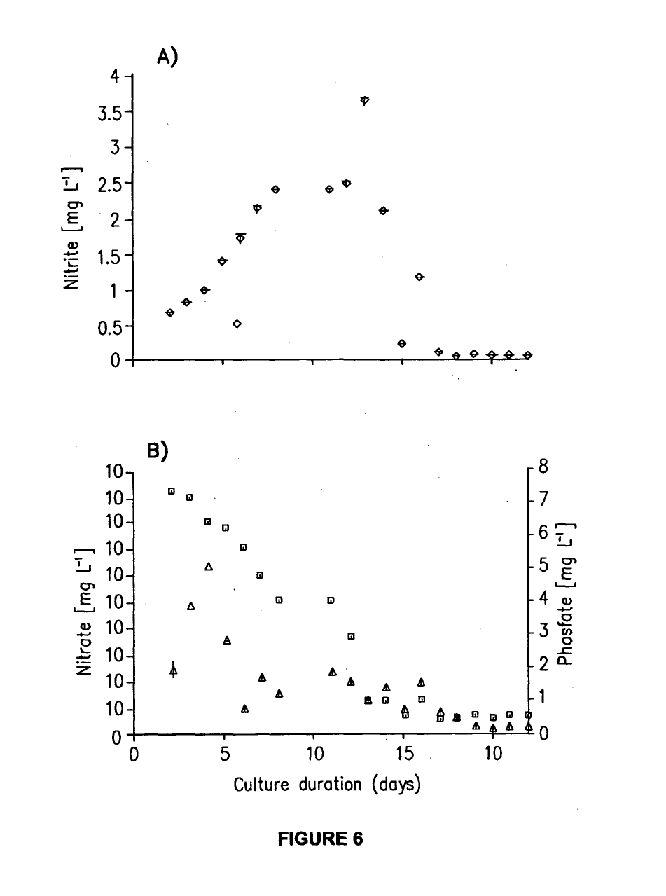

FIG. 6 is a graph showing the time course of nutrient concentrations in the bag cultivation chamber. A) nitrite, B) nitrate (red squares) and phosphate (black triangles). Average±standard deviation, n=3.

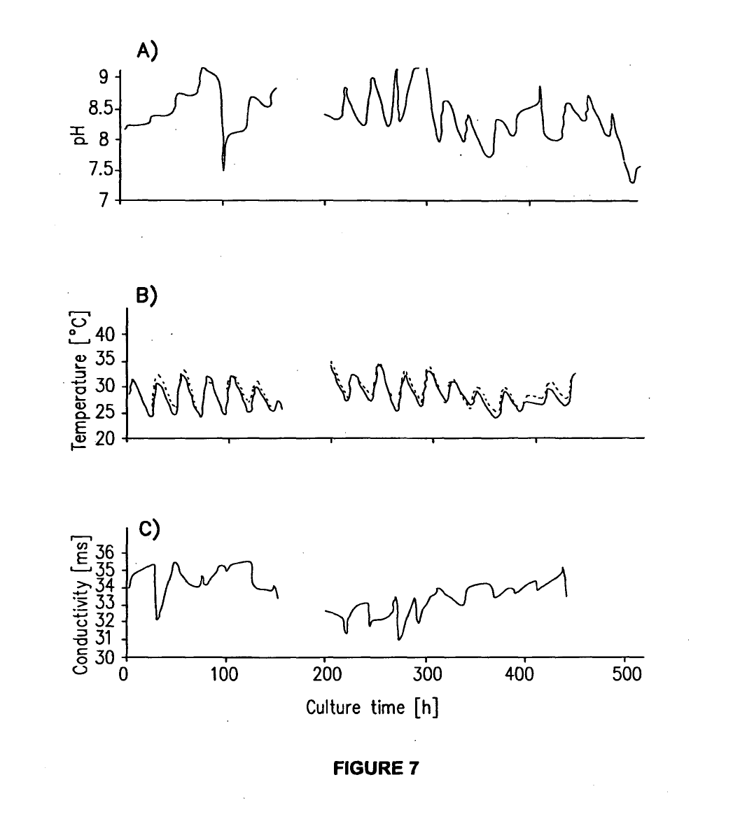

FIG. 7 is a graph of the fluctuation of A) pH, B) temperature; and C) conductivity over culture time of Nannochloropsis oculata in the bag. WP-81: handheld TPS pH- and conductivity-meter, manual: handheld thermometer.

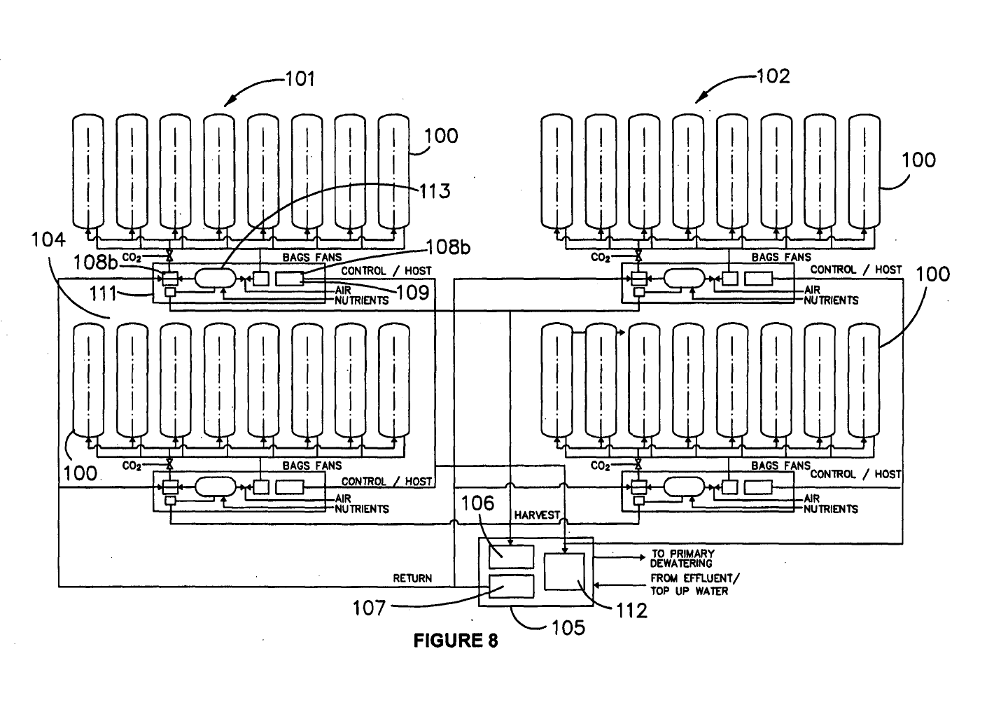

FIG. 8 is a schematic diagram illustrating a series of cultivation chambers with central control unit.

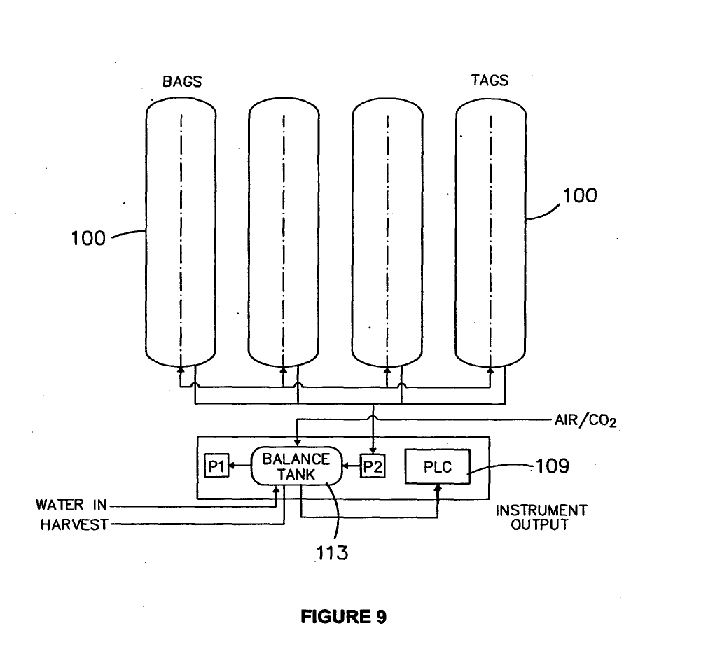

FIG. 9 is a schematic diagram illustrating a plurality of cultivation chambers in series with an enclosed cultivation chamber.

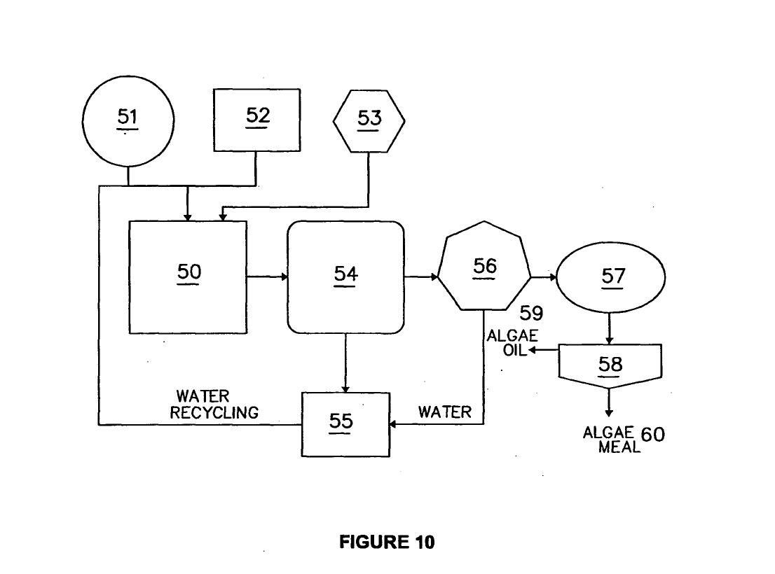

FIG. 10 is a schematic diagram illustrating carbon capture and recycling process overview.

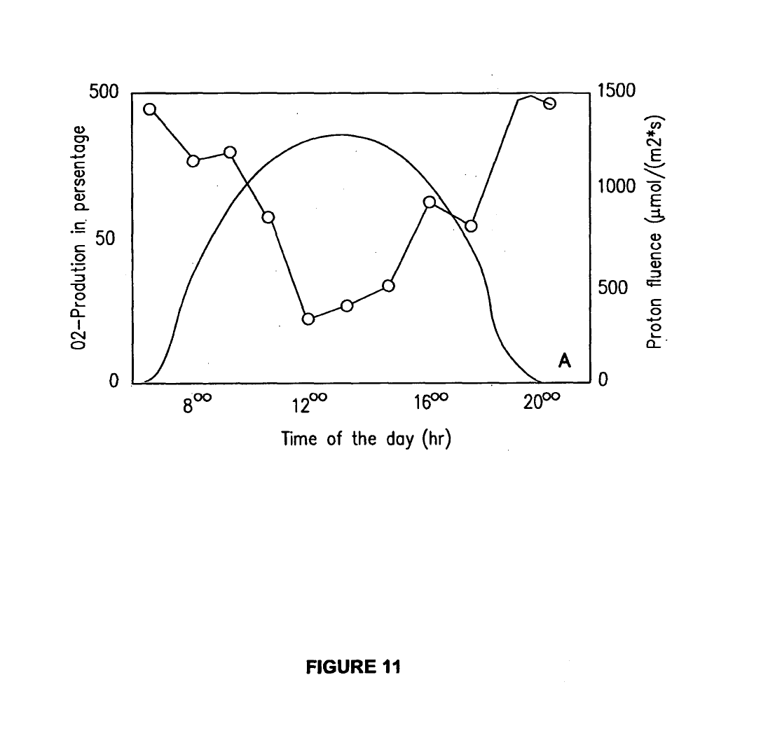

FIG. 11 is a graph illustrating Photo-inhibition demonstrated via O2 production over a 24 hour period in which the left axis is O2 production in percent the base axis is time of day and the right axis is proton fluence (μmol/(m2*s)

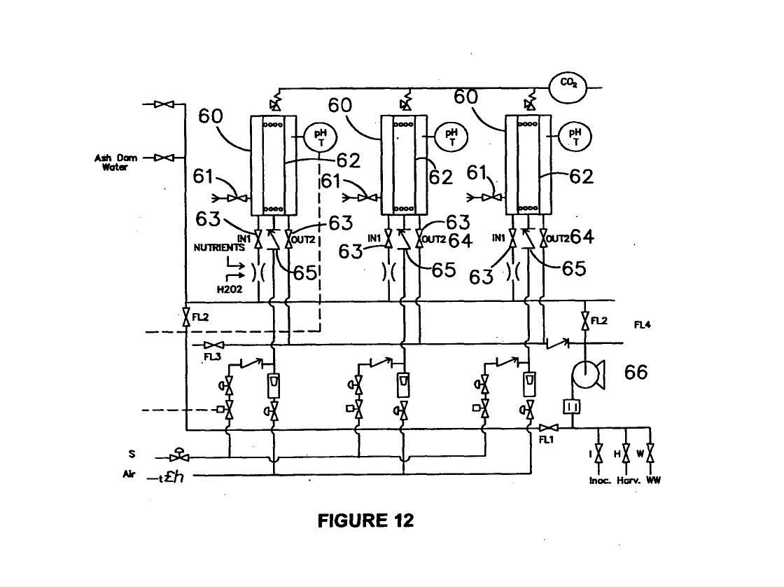

FIG. 12 is a schematic diagram illustrating a number of vertical growth columns

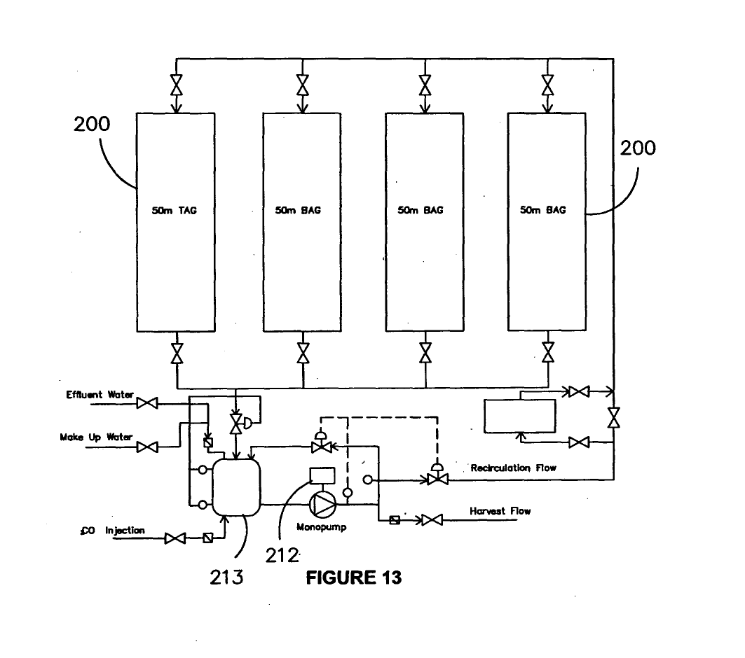

FIG. 13 is a schematic diagram illustrating a plurality of cultivation chambers in series with an enclosed cultivation chamber as shown in FIG. 9 showing the control loops and inputs into the system in detail and

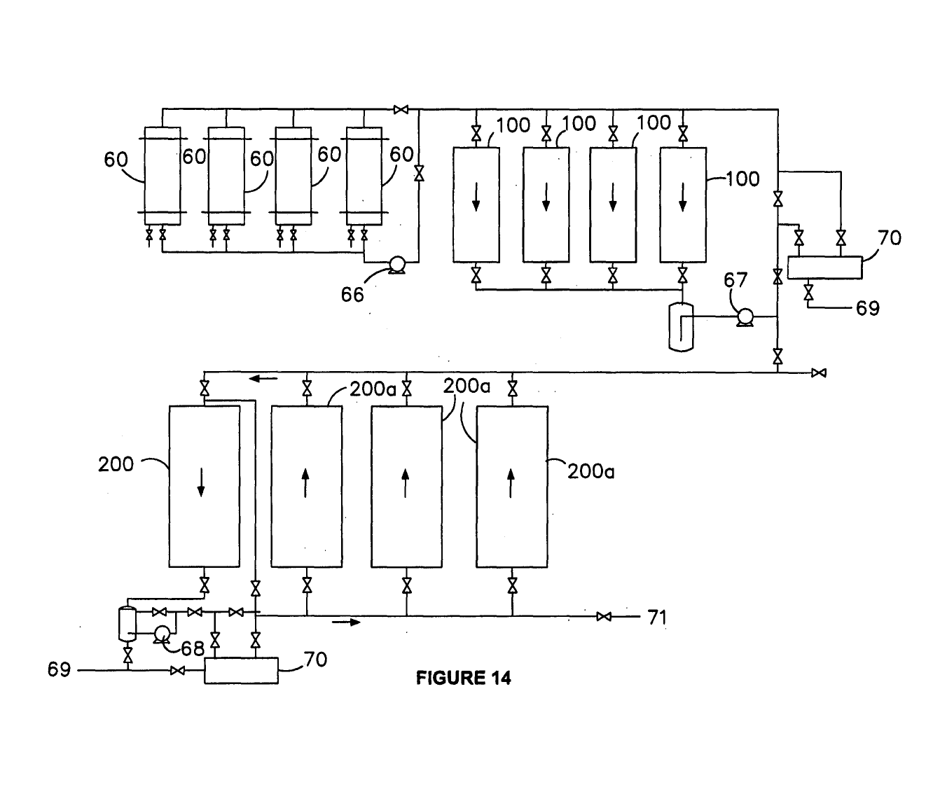

FIG. 14 is a schematic diagram of a biological cultivation system using a number of different sized cultivation chambers.

### DETAILED DESCRIPTION OF THE EMBODIMENTS

### Example 1

A chamber for the cultivation of photosynthetic organisms was created using a bag culture system as shown in FIG. 1. The cultivation chamber 1 includes a flexible bag (1) containing a culture medium for growing algae (2); gas outlet (3); fan (4); gas inlet (5); cultivation medium outlet (6); and cultivation medium inlet (7).

The operation of bag cultivation chamber 10 is as follows:
1 A fan (4) inflates the empty cultivation chamber (without culture medium (2)) to operational volume, with all excess pressure exiting through the gas outlet (3). The fan is continuously running so as to ensure the bag (1) does not deflate.
2 The empty cultivation chamber is inoculated with 10 000 l of microalgae culture (0.2% algae) produced in a separate photobioreactor and topped up with 10 000 l filtered and treated recycled saline waste water.
3 CO₂ is injected continuously during daylight hours through the gas inlet (5). The microalgae absorb the required quantities of CO₂ and the excess is released through the always open gas outlet (3).
4 An additional 20 000 l of recycled saline waste water is added, bringing the total capacity to 40 000 l of culture medium.
5 This process continues for another 24 hours until total harvesting capacity reaches 100 000 l. At this stage, the level of the culture medium (2) in the cultivation chamber is 60 cm.
6 After the algae have reached maximum harvest capacity (72 hours), 50 000 l is harvested from the cultivation medium outlet (6).
7 50 000 l of recycled saline waste water is returned to the cultivation chamber via the cultivation medium inlet (7), bringing the total culture medium volume back to 100 000 l.
8 The harvesting and return cycle repeats once every 24 hours, while maintaining continuous CO₂ injection during daylight hours.

### Example 2

The bag cultivation chamber described in Example 1 was modified as shown in FIG. 2. In this embodiment, the cultivation chamber 20 includes a flexible bag (11) containing a culture medium for growing algae (12); gas outlet (13); gas bubbling tracks (14) with pinprick holes (15); gas inlets (16); cultivation medium outlet (17); cultivation medium inlet (18); draining outlet (19) and float valve (10a) to regulate ports (17), (18) and (19).

The operation of bag cultivation chamber 2 is as follows:
1 Cultivation chamber 20 inoculation follows the same procedure as Example 1 steps 2, 4 and 5 to bring the harvesting capacity to 100 000 l within 72 hours.
2 CO₂ is pre-mixed with a high efficiency particulate air (HEPA) filtered air stream and fed through gas inlets (16) to gas bubbling tracks (14). These tracks are pinpricked (15) at suitable intervals to allow even air/CO₂ distribution along the length of the bag cultivation chamber. This bubbling operates continuously, with the CO₂ component reduced overnight.
   The air/CO₂ injection acts to slowly inflate the bag (11) and maintain circulation of the algae in the culture medium (12). Excess pressure is released through the one-way valve regulated gas outlet (13). This creates a closed loop system to minimise the contamination risk.
3 After the 72 hours of culturing the microalgae, 50 000 l is harvested from the ball-valve-regulated (10a) cultivation medium outlet (17), which is positioned at 30 cm in height. Once the cultivation medium reaches 30 cm, a signal is sent to the automation system that the cultivation chamber is at 50 000 l capacity.
4 50 000 l of treated recycled saline or freshwater waste water is returned to the cultivation chamber via the ball valve-regulated (10a) cultivation medium inlet (18), sending a back pressure signal to the automation system that the cultivation chamber is now at 100 000 l.
5 The ball valve-regulated (10a) draining outlet (19) allows the complete draining of cultivation chamber in case of contamination or for a regular cleaning routine. The remaining cultivation medium is either drained to the harvesting system for processing or, in the case of contamination, to the UV treatment system.

### Example 3

The bag cultivation chamber described in Example 1 was further modified as shown in FIG. 3. The cultivation chamber 30 includes a flexible bag (21) containing a culture medium for growing algae (22); gas outlet (23); gas bubbling tracks (24) with pinprick holes (25); gas inlets (26); cultivation medium outlet (27); cultivation medium inlet (20b) with pressure sensor (18) and ball valve (20a).

The operation of bag cultivation chamber 30 is as follows:
1 Cultivation chamber 30 inoculation follows the same procedure as Example 1 steps 2, 4 and 5 to bring the harvesting capacity to 100 000 l within 72 hours.
2 CO₂ is pre-mixed with a high efficiency particulate air (HEPA) filtered air stream and fed through gas inlets (26) to gas bubbling tracks (24). These tracks are pinpricked (25) at suitable intervals to allow even air/CO₂ distribution along the length of the cultivation chamber. This bubbling operates continuously, with the CO₂ component reduced overnight.
   The air/CO₂ injection acts to slowly inflate the bag (21) and maintain circulation of the algae in the culture medium (22). Excess pressure is released through the one-way valve regulated gas outlet (23). This creates a closed loop system to minimise the contamination risk.
3 After the 72 hours of culturing the microalgae, 50 000 l is harvested from the cultivation chamber through the ball valve-regulated harvesting outlet (27). The required volume is determined by measuring volume in reference to a pressure head sensor (28).
4 50 000 l of treated recycled saline or freshwater waste water is returned to the cultivation chamber via the ball valve-regulated (29) cultivation medium inlet (20b) with pressure head sensor (28), sending a back pressure signal to the automation system that the cultivation chamber is now at 100 000 l.

Further detail (in top view) of a gas bubbling setup that may be included in the modified cultivation chamber is provided in FIG. 4. This figure shows gas bubbling tracks (35) in the base of a bag cultivation chamber having a gas inlet (31) with compression fitting (32), a conduit (33) to transport the gas to restrictive flow orifices (34) and the end of each track (35) and pinprick holes (36) to allow the exit of gas.

This figure shows six gas bubbling tracks (35) with pinprick holes (36) which are fed with gas introduced through the gas inlet (31) and compression fitting (32) via a gas conduit (33) and restrictive flow orifices (34) at the end of each track. The restrictive flow orifices serve to divide the gas flow evenly between the air distributor vanes. The flow rate of the gas through the gas inlet is approximately 100 kg/hr and through the restrictive flow orifice 17 kg/hr.

### Example 4

The growth of the microalga Nannochloropsis oculata was tested using the bag cultivation chamber described in Example 1. This culture bag was 10 m in length and 3 m in width and fitted with a six-bladed fan at one end to keep the bag inflated and drive evaporation. Along the top of the bag, four holes (13 cm diameter) allowed hot air and vapor to escape. This evaporation assisted in maintaining the algal culture at more stable temperatures.

In this trial, both freshwater and filtered marine aquaculture waste (A3) water was added to the culture to account for salinity increases and evaporative loss of liquid. The bag cultivation chamber was filled to approximately 0.30 m in depth, resulting in a final culture volume of slightly less than 9 m³. The algae were cultivated in sea water than had been filtered through 20 μm, 5 μm and 1 μm filters.

Aeration and CO₂ enrichment was provided through tubing designed for the gas diffusion via delivery into liquid media. This tubing had an outer diameter of 25 mm, an inner diameter of 10 mm and a porous wall of 7.5 mm thickness.

The bag cultivation chamber system was inoculated with Nannochloropsis oculata with an apparently low cell concentration of 2.1×10⁴ cells mL⁻¹ and not filled up to full capacity volume. Already after 24 h, cell densities had increased dramatically, indicating time requirements for complete mixing of inoculate and culture medium (incomplete mixing affects correct determination of cell concentration), and the bag was filled up to its maximum depth on day 2. The growth of the culture up to the harvest of the algae on day 20 is shown in FIG. 5.

### Nutrient Consumption

There was a steady increase in nitrite from the day of inoculation (0.5 mg L⁻¹) to day 8 (2.5 mg L⁻¹) (FIG. 6 A). After a few days at a steady concentration, nitrite peaked at 3.7 mg L⁻¹ on day 13, and then was rapidly utilized. Within a few days, nitrite was depleted and remained so until the culture crashed. Nitrate was a high 90 mg L⁻¹ at the beginning of the period, and was steadily being utilized (FIG. 6 B). From day 13, nitrate concentration remained stable around 10 mg L⁻¹. There was an increase in phosphate the first few days (through addition of filtered A3 water to top the system up) (FIG. 6 B). From day 3, phosphate was being noticeably assimilated and fluctuated between 2 mg L⁻¹ and totally deplete. No nutrients were added to the bag system, however fresh filtered A3 water was regularly added along with freshwater to compensate for evaporation. The added A3 water accounts for the regular, small increases in nutrient concentrations.

### Physical and Chemical Parameters

In the culture, pH quickly rose to over 9 in the first three days (FIG. 7). After day 3, a CO₂ supply was connected and pH could now be regulated by adding CO₂ when a value above 8.4 was recorded.

Photosynthetic activity was high in the bag in the beginning of the period, with rapid changes in pH due to uptake of CO₂ during photosynthesis, leading to large fluctuations in pH.

Temperature fluctuated in diel rhythm, with the highest temperatures measured in the afternoon (4 pm) (FIG. 7 B). Similar to the tank system, temperatures rarely rose above 30° C., and were quite stable.

Conductivity in the bag fluctuated between 32 and 36 mS due to evaporation, and both freshwater and additional filtered A3 water was regularly added (FIG. 7 C).

The carbon capture and recycling process according to the present invention will be described with reference to FIGS. 8, 9 and 10. FIGS. 8 and 9 show an embodiment of a cultivation system in accordance with the invention. The system includes a plurality of cultivation chambers 100. The cultivation chambers 100 are shown as arranged in parallel in four cultivation sections 101, 102, 103, 104 which are also connected in parallel to a pumping station 105. The pumping station 105 includes a harvest pump 106 and return pump 107.

Each cultivation section is provided with a valve manifold assembly 111 including metering 108a and 108b to monitor the rates of flow of CO₂, nutrient and media (water) to and from each cultivation section of cultivation chambers 100 and a programmable logic controller (PLC) 109 to control the flow rates to optimize growth of the photosynthetic organisms in the cultivation chambers 100. A balance tank 113 is also provided on the supply side to ensure that a head is maintained for pumping. The operation of each valve manifold assembly 111 form each cultivation section is controlled by a master controller 112.

The control unit 105 may include one or more of:
(a) a mobile or fixed balance tank 113 and/or sampling point with a reservoir capacity of water growth medium used to produce algae.
(b) process units, circulation, harvesting and make-up water pumps 106, 107. These pump from the control unit 112 to the cultivation chambers either directly via pipes or via a valve manifold assembly. 111
(c) a tank or reservoir volume 113 that may act as a dosing point for nutrients, chemicals and CO₂, data collection point for instrumentation used for process collection and measurement. The tank or reservoir volume 113 may allow a period of darkness to allow the algae to have rest period
(d) a discrete power supply interface for the above function requirements
(e) a process controller device 109 ie, discrete 10 virtual PLC and conventional PLC as well as SCADA device type equipment

In an alternative embodiment (FIG. 13), the control unit may include one or more of
(a) a fixed balance tank and/or sampling point with a reservoir capacity of water growth medium used to produce algae. This may be a fixed above ground 4-sided water tight structure 200 with bulkheads that will allow the secure mounting of valves, pumps and other ancillary equipment used to grow algae. This may be covered with a light impermeable cover to either block light from the algae in medium or with a light permeable cover to allow light to reach the algae depending upon process requirement.
(b) process units, circulation, harvesting and make up water pumps 212. This will pump from the rigid cultivation chamber 200 to the bulk cultivation chambers 200 either directly via pipes or via a valve manifold assembly.
(c) a tank or reservoir volume 213 that will act as a dosing point for nutrients, chemicals and CO₂, data collection point for instrumentation used for process collection and measurement.
(d) a discrete power supply interface for the above function requirements
(e) a process controller device ie, discrete IO virtual PLC and conventional PLC as well as SCADA device type equipment
(f) an evaporative cooling assembly (not shown) mounted internally to cool an entire maniple (group) of parallel cultivation chambers e.g. via solar radiation will be routed through the rigid chamber and cooled via evaporation water fall and air movement.
(g) a means of air movement e.g.; blower mounted to the bulkhead of the rigid chamber.
(h) the rigid chamber 200 may also incorporate both water recirculation and/or air bubbling reticulation devices allowing both air and water to
   a) circulate algae for process flow to ensure homogeneous distribution of the algae throughout the growth system
   b) keep algae in suspension to promote homogeneous algae distribution through the growth medium water and prevent stratification of algae
   c) act as venturi delivery system for additional air or CO₂

The control unit may further include a drive controller(s) to control the pumping rates of the culture medium. Preferably the drive controller(s) are variable speed drive controllers (VSP). Variable speed drives to control pumps both AC and DC powered allow variable pumping rates of algae growth medium for use in process control. The pumping rates of water may be varied upon growth requirements or harvesting/make up Input/output (I/O) Variable Frequency Drives (VFD), Pulse width modulated (PWM) and/or Vector type pumping drive controllers. DC drive controllers can be either variable voltage control or with pulse width modulation.

The above mentioned VSD based controller operates on variations of an AC input to DC bus to AC output. Permutations may include:
(a) AC input to DC bus via rectifier of an inverter that is used for leveling voltage feeding an AC output—This will allow AC generation sources for renewable energy such as 3 phase wind turbines or conventional mains power to drive or power and control an AC powered electrical motor, pump, blower or frequency emitter. This can be used but not is limited to operate a pump, blower or lysing operation.
(b) A direct DC input such as solar panels and/or DC wind turbines to the DC BUS of an inverter that will invert DC to AC to drive an AC powered electrical motor. This can be used but is not limited to operate a pump, electric blower, process controller or any other device associated with algae production/harvesting.
(c) A combination of both AC and DC inputs jointly to function as above to drive an AC powered device such as a pump, power supply, process controller or algae lysing device that relies on either a source of renewable energy DC or conventional mains power (AC?).
(d) A combination of (a), (b) and (c) I allowing an inverter based device to drive either a DC or AC PWM output for algae lysing (cracking).

FIG. 10, shows the process flow of production and harvesting of algae and other photosynthetic organisms. The Biological Algae Growth System (BAGS) 50 are initially filled with fresh/salt water 51 in line with nutrient dosing, from a dosing unit 52. These bags are then inoculated from an existing source of algae at harvesting density.

CO₂/flue gas 53 to aid biomass growth and filtered air for circulation and dissolved O₂ off gassing was transferred to the BAGS during the algae's growth cycle.

Once harvesting algae density is attained (up to 1.0 wt % but typically 0.2 to 0.7 wt %), the BAGS are harvested and transferred to the dewatering stage 54.

The dewatering stage transfers the centrate/filtrate water to a treatment plant prior to recycling the water back to the BAGS via nutrient dosing and water top-up.

The algae concentrate from the dewatering stage proceeds to a thickening stage 56 to further concentrate the algae.

This concentrate may then be transferred to lipid extraction 57 and product separation 58 to attain high quality algae oil 59 and meal 60 for further product treatment and distribution.

### Functional Requirements

The key functional requirements for the CRS (computerised reticulation system are as follows:
Provide sufficient flow rate to enable optimal growth rate of algae biomass
To house the control systems and power source for all local valves, pumps, instruments, cooling systems and reticulation systems.
Ensure efficient balance of flow during the algae recirculation phase of operation.
Ensure continual pump prime is maintained for circulation and harvest phases.
Integrated and controlled carbon dioxide (CO₂) injection manifold to ensure optimal growth of algae by preventing carbon limitation.
Integrated gas escape mechanism whereby waste gas (e.g. dissolved O₂) can be exhausted from system.
Ensure gas is discharged appropriately according to power station and DERM requirements.
Inclusion of algae broth temperature control mechanism to maintain the algae within prescribed growth limits.
Protection protocols and systems installed to ensure minimal algae contamination possible from other microorganisms.
Integration with all in field instrumentation to gather relevant growth data.
Designed sufficiently stable and robust to handle continual outdoor exposure.
Minimise energy usage.
Ease of maintenance, particularly ease of cleaning.
Ease of connection and disconnection of skid and its individual plumbed and electrical components

### Algae Growth System Design

A single cultivation section is illustrated in FIG. 9 consists of a manifold of three 50 m BAGS 100 and one 50 m TAGS. 100 The BAGS 100 are made from a translucent polypropylene, while the TAGS consist of a transparent Laserlite™ covered and lined above-ground pond. The BAGS are inflated by small electrical motor driven fans. Vent openings on the BAGS 100 and TAGS 100a permit free release of excess gases and excess dissolved oxygen (O₂) produced during the photosynthetic growth phases of the algae.

### Process

The initial water injection or water make up for the BAGS/TAGS, as well as the inoculation algae stream, will be transferred from elsewhere on site to the balance tank.

Pump P1 is used to pump from the balance tank 113 into the combined manifold and then along the length of the four growth vessels (via a perforated sparge bar inside the algae solution).

Pump P2 is then used to pump from the growth vessels back to the balance tank 113. Circulation is maintained via the loop: VESSELS>P2>BALANCE TANK>P1>VESSELS.

Filtered air is introduced during the circulation phase to remove dissolved O₂ produced during the growth phase. This could be into the buffer tank, directly into the growth systems or both.

CO₂ is introduced during the circulation phase, directly into the liquid stream before it enters the vessels. This is modulated according to the photosynthetic requirements of the algae.

Nutrients are also dosed into the balance tank during the circulation phase according to the photosynthetic requirements of the algae.

When harvesting the algae the biomass is dewatered resulting in higher concentration of organisms per unit volume, pump P3 transfers the algae solution from the balance tank to the dewatering system on site for product concentration.

### Interface

The following connections interface with the CRS (controlled release system):

### Inputs

Water/inoculation line for fresh/salt top-up water and inoculation from other algae growth systems.
Harvest line from BAGS/TAGS to skid
Filtered air @ 0.4 bar for dissolved O₂ off gassing.
CO₂ @ 9 bar for algae growth.
Control inputs from master PLC (programmable logic control).
Power for skid equipment, associated valves and instrumentation and BAGS fans.

### Outputs

Harvest line for harvest of algae biomass when at harvesting density.
Return line from skid to BAGS/TAGS
Instrumentation and pump and valve status outputs

### Display Facility

### Concept

The multi-component growth system illustrated in FIG. 8.

### Process

The individual circulation process occurs as outlined above, only with a greater volume of BAGS. The CRS is intended to control up to eight BAGS in the display-scale growth system, with a master control system monitoring the outputs from each unit. All harvested algae and return water is transferred via the master system to distribute to the local CRS.

### System Data

All system data is based on the research-scale algae growth system.

### Reticulation

### Growth System Specifications

Specifications for the algae growth system:

| | Design | |
|---|---|---|
| Parameter | Value | Comments |
| BAGS length, m | 50 | |
| BAGS width, m | 3.4 | |
| BAGS depth, m | | |
| Normal working depth | 0.3 | Design BAGS systems to allow trial |
| Maximum working depth | 0.6 | operation up to 0.6 (optimal height) |
| Inflated bag height | 0.8-1.1 | |
| Number of BAGS in | 4 | |
| manifold | | |
| BAGS volume range, kL | 50-100 | For one BAGS (0.3-0.6 m depth) |
| Total BAGS volume | 200-400 | For a manifold of four BAGS |
| range, kL | | |
| Broth salt content | 0-50 | High end during evaporation loss |
| (ppt salt) | | |
| Max. daily evaporation | 5000 | Utilising cooling fan |
| loss, L | | |
| Operational water | 20-40 | |
| temperature (° C.) | | |

### Turnover Rate

A number of factors influence the required growth system turnover (one complete cycle of system) time. These include the circulation rate of the algae, the response time of the critical growth parameters and the photosynthetic cycle over the growth period. Below is a discussion of how these factors affect the turnover rate for sizing of the circulation pumps on the skid.

### Experimental Results to Date

The Photo Bio Reactor (PBR) based on 10 metre BAGS has maintained stable algae growth at flow rates between 3-5 volume turnovers per hour.

### Algae Circulation Rate

It is preferred to keep the algae circulating inside the BAGS at an approximate rate of 1 Hz (1 cycle per second). This permits the algae to receive a more even distribution of light, nutrients and CO₂ as well as ensuring the algae does not settle to the base of the growth system and develop bio fouling.

Two methods of creating sufficient biomass circulation patterns are via air bubbling or high velocity fluid injection.

### Critical Growth Parameters

Nutrient addition: there is a requirement to control the nutrient injection into the broth accurately as this has a significant effect on lipid production. Maintaining the nutrient feed on the edge of starvation has been suggested to increase the overall lipid content of the given species.
Complete nutrient consumption may occur in as little as 12 hours, and algae may be starved for a period of approximately 1 to 5 hours before harvesting to promote lipid production. An estimated acceptable response time for nutrient control is 30-60 mins. (i.e. at least 1-2 volume turnovers per hour)
CO₂ addition: CO₂ is added to the broth for two primary purposes: to act as a controlling mechanism for the pH level (to bring down a high pH add more CO₂) and to ensure that the algae are not carbon limited during the photosynthetic active growth period. It is important to monitor the pH and carbon regularly to ensure maximum cell reproduction and to minimise the harvesting period. An estimated acceptable response time is 15 mins. (i.e. at least 4 volume turnovers per hour).
Dissolved O₂ reduction: an increase in fluid movement can aid in the reduction of dissolved O₂ by increasing the air to water surface contact area, which is essential to ensure that the algae does not experience oxygen super saturation (poisoning effect). An estimated acceptable flow rate is 1 volume turnover per hour.

### Photosynthetic Response

The diurnal cycle (day/night) is an important control factor in the turnover rate calculations. During the night and period of low photosynthetic response (heavy clouds, shading etc) the following changes apply:
CO₂ consumption drops to near zero
Respirated O₂ output decreases
Nutrient consumption decreases
Growth rate slows (night)

Therefore parameter response times and circulation rates become less critical and there is not the same turnover rate requirement during these periods. At night, the main variable becomes the risk of biofouling due to lack of circulation of the algae broth. This implies that the circulation pumps on the CRS will require either a large range of flow rates, or two separate pumps (one for high speed day cycle and one for low speed night cycle).

The other component of the growth cycle that will affect the above changes is photo-limitation and photo-inhibition (FIG. 11), which occurs during period of high fluence (light exposure). This is a photo-protective mechanism of all algae species (varies by species) that impairs the photo systems to protect the photosynthetic apparatus. Essentially it means that photosynthesis is also limited during periods of very high light intensity (i.e. during the middle of the day). This further supports the notion that a range or two separate circulation flows may be useful, to allow adequate control of critical parameters during periods of high photosynthetic activity while minimising energy consumption the remainder of the time.

### Circulation Flow Rate Summary

| | Design | |
|---|---|---|
| Parameter | Value | Comments |
| PBR trial, volume turnover per hour | 3-5 | |
| Previous BAGs trials, volume turnover per | 1-2 | |
| hour | | |
| Algae circulation, volume turnover per hour | [TBC] | |
| Nutrient addition, volume turnover per hour | >1-2 | |
| CO₂ addition, volume turnover per hour | >4 | |
| O₂ reduction, volume turnover per hour | >1 | |
| Total BAGS volume, kL | 200-400 | For a manifold |
| | | of four BAGS |
| Flow rate at 0.3 m depth, kL/min: | | |
| 1 volume turnover per hour | 3.4 | |
| 5 volume turnovers per hour | 17 | |
| Flow rate at 0.6 m depth, kL/min: | | |
| 1 volume turnover per hour | 6.8 | |
| 5 volume turnovers per hour | 34 | |

### Harvesting Flow Rate

The flow rate for the harvesting pump must also be considered. It has been proposed to harvest 25-50% of the BAGS volume every 24 or 48 hours. The time over which the desired BAGS volume must be harvested is approximately 8 hours; this is a trade-off between the harvesting system size required and the length of time an operator must be present on site.
For BAGS at a depth of 0.3 m, to harvest 25% over 8 hours the required flow is 6.25 kL/hr; at 50% the required flow is 12.5 kL/hr.
For BAGS at a depth of 0.6 m, to harvest 25% over 8 hours the required flow is 12.5 kL/hr; at 50% the required flow is 25 kL/hr.

### Head Requirements

In order to circulate the algae, low head high flow pumps are required.

Some approximate calculations were carried out on Pump 1 to determine rough pipe sizes and head requirements for the various flow rates:

| Flow rate | Manifold size (mm) | Sparge Bar size (mm) | Head (m) |
|---|---|---|---|
| 400 kL/hour | 280 | 140 | 2.6 |
| 800 kL/hour | 355 | 180 | 3.4 |
| 1600 kL/hour | 500 | 250 | 3.1 |

Assumptions: minimum tank level of 0.6 m, PE piping, all BAGS operating at 0.6 m

Note that the approximate pipe diameter for a flow of 1600 kL/hour (4 volume turnovers per hour) is almost half of the water depth in full BAGS and almost the entire depth in harvested BAGS.

Similar calculations were carried out on Pump 2. As an example, at 400 kL/hour with the same pipe sizing given above (280 mm manifold, 140 mm sparge bar) approximately 1.4 m head is required to transfer the algae from BAGS at 0.3 m depth to a tank with a top level of 0.3 m. Obviously as the height of the tank top level increases, the head requirements will increase accordingly. Care will need to be taken to ensure there is adequate suction head available for the pump. For gravity flow with these pipe sizes, the tank top level would have to be at least 1.4 m above the top water level of the BAGS.

Care will also need to be taken in pump selection to ensure the algae are capable of handling the shear forces involved in pumping.

### Balance Tank System

An internal balance tank on the CRS may be used:
To maintain sufficient head for pump P1 and balancing circulation flows between pumps P1 and P2
Point for nutrient monitoring and injection
Point for instrumentation package to interface
Harvesting point
Potential for dissolved O₂ off gassing
Sampling point for manual analysis

Note that a mixing or circulation arrangement may be required to ensure there is no settling of algae. Valves will also need to be carefully considered, as there will be a requirement for two-way pressure adjustments while preventing contamination from entering the tank.

### Growth Parameters

### Nutrient Addition

For the research plant, the required nutrients will be supplied in the form of pre-prepared and sterilised solutions—one for nitrate and the other for phosphate.

Two separate dosing pump systems will be used, allowing the ratio to be readily adjusted. Dosing could be directly into the balance tank on the BAGS skid or in line with the fluid flow, with the dosing flow automatically proportioned based on broth flow rate and nutrient monitoring.

The required nutrient concentrations are:

| Nutrient | Design Point | Allowable Range |
|---|---|---|
| Nitrate | 0-30 mg/L | 0-100 mg/L |
| Phosphate | 0-10 mg/L | 0-50 mg/L |

### CO₂ and Air Injection

As has been previously mentioned, CO₂ is added to the algae solution for two primary purposes: to ensure the algae are not carbon limited during the period of photosynthetic activity and to act as a controlling mechanism for the pH level. This may be supplied directly from a pressure vessel containing pure CO₂, however this may also come from the flue gas and will thus be heavily diluted.

Direct injection into the algae solution circulation flow via a venturi arrangement may be the most effective method for increasing dissolved CO₂. An alternative method is to bubble the CO₂ through the solution in combination with the air bubbling described below.

As has also been previously mentioned, excess dissolved O₂ may be removed from the water as this can poison the algae. One method to assist with this is simply fluid circulation, which increases the air to water surface contact area and thus allows more O₂ to come out of solution.

A second method to assist with dissolved O₂ reduction is bubbling air through the algae solution from the base of the BAGS. Provided that enough dissolved CO₂ remains in the solution (CO₂ can be readily displaced by oxygen and nitrogen if air is bubbled through water), air bubbling through the BAGS may help to draw dissolved oxygen out of the solution. Note that the transfer of molecules from a gaseous form to a dissolved form is dependent on solubility and relative concentration levels; there is an equilibrium condition between the gaseous and dissolved forms. For example, nitrogen gas molecules can readily displace CO₂ gas molecules. To maintain equilibrium this causes more dissolved CO₂ to come out of solution.

Finally it has also been previously mentioned that the algae should be kept circulating inside the solution to receive a more even distribution of light, nutrients and CO₂ as well as ensuring the algae do not settle out of suspension. A second advantage of air bubbling (aside from dissolved CO₂ reduction) is that it may be used to assist in this internal circulation of algae within the BAGS.

### Temperature Control

A temperature control system may optionally be included.

### System Management

### Operation

Operational specifications:
24 hour continuous pump operation

### Control Requirements

Valve and pump requirements and in field solenoid valves

The list of associated instrumentation to interface with CRS PLC:

| | | | | | | |
|---|---|---|---|---|---|---|
| | | | Sensor | | | |
| | | Desired | Spec. | | Dependant | Control |
| Data | Units | Range | Range | Purpose | Relationships | Mechanism |

Physiochemical Data

| | | | | | | |
|---|---|---|---|---|---|---|
| Dissolved O₂ | % | 80-130 | 0-300 | Avoid oxygen | | Fluid |
| | | | | super saturation | | recirculation |
| | | | | | | Bubbling |
| Dissolved CO₂ | g/L | 0.05 g/L | | Control pH | pH | Throttle CO₂ |
| | | | | Photosynthesis | Dissolved O₂ | injector |
| | | | | | (photosynthesis | |
| | | | | | dependant) | |
| Free CO₂ | μL/L | | | Monitor CO₂ | Dissolved O₂ | Throttle CO₂ |
| | | | | uptake by algae | (photosynthesis | injector |
| | | | | | dependant) | |
| pH | Value | 7.8-8.2 | 3-12 | Broth acidity | Dissolved CO₂ | CO₂ injection |
| | | (SW) | | detection | | |
| | | 6.4-7.4 | | | | |
| | | (freshwater) | | | | |
| Temperature - | ° C. | 25-30 | 0-50 | Maintain ideal | | Cooling |
| Liquid | | | | growth temps | | system |
| Temperature - | ° C. | 25-30 | 0-60 | Maintain ideal | | Cooling |
| Air | | | | growth temps | | system |
| Conductivity | μS/cm | 5-7 × 10⁴ | 0-10 × 10⁴ | Monitor salinity | Temperature | Dose in |
| | | Include | | of water | | additional fresh/ |
| | | freshwater | | | | salt water |
| | | range | | | | |
| Weather Data | | | | Temp | | |
| | | | | Humidity | | |
| | | | | Light levels | | |
| | | | | Wind speed | | |
| | | | | Rain gauge | | |

Nutrient Data

| | | | | | | |
|---|---|---|---|---|---|---|
| Nitrate | mg/L | 0-40 | 0-100 | Consumption | All | Dosing pump |
| | | | | Top ups | | |
| Nitrite | mg/L | 0-30 | 0-100 | Consumption | All | Dosing pump |
| | | | | Top ups | | |
| Phosphate | mg/L | 0-10 | 0-50 | Consumption | All | Dosing pump |
| | | | | Top ups | | |

Biomass Detection

| | | | | | |
|---|---|---|---|---|---|
| Turbidity | % trans at | 30-80% | 0-100% | Determine | All |
| | 750 nm | rel. to | | algae cell count | |
| | | salt/fresh- | | | |
| | | water water | | | |
| | | medium | | | |
| photosynthetic | μmol/(m²*s) | | | Determine | |
| photon flux | | | | algae cell count | |
| density | | | | (higher | |
| | | | | precision) | |
| | | | | CO₂ uptake | |

### Vertical Growth Column

FIG. 12 illustrates an embodiment of an array of vertical growth columns 60 used in conjunction with the substantial horizontal cultivation chamber system of the present invention. Each column comprises a substantially cylindrical outer conduit 61 and a substantially cylindrical inner conduit 62 arranged vertically. The inner and outer conduit are fluidly communicating to enable growth media and algae which make up the algal slurry to circulate between. The growth columns 60 are provided with fluid inlets 63 and fluid outlets 64 for the introduction of growth media and an inoculate of algae and the removal of algal slurry. The columns 60 are also provided with gas inlets 65 for the introduction of CO₂ and air and gas outlets for the removal of gas. The gas outlets are provided with CO₂ sensors to monitor the CO₂ content on the outgoing gas.

A pump 66 is provided to circulate the fluid to the columns. Once in the columns, the inflow of gas into the centre of the growth column causes the algal slurry to rise up to the top of the inner conduit before passing to the outer conduit where it descends the column. An alternative arrangement may be to add the gas to the outer conduit and have the algal slurry descend in the inner conduit. The choice of arrangement will depend on the growth rates of the algae, the density of the algae during circulation and the light transmissivity of the algal slurry at different stages of circulation. Once the algal slurry has reached a suitable density then it is removed through outlets 67 and either used as a product or used as an inoculate in the cultivation chambers of the growth system.

While the growth columns are shown as connected in parallel, the columns can be connected in series and optionally used as a stand alone growth system if the resulting growth rates in the columns are sufficiently high and sufficient product can be grow for the purposes required. The applicant has found that algal densities of up to 30-75 grams DW per square meter of media can be sustained in the vertical growth columns compared to approximately 20-35 grams DW per square meter in the cultivation chambers discussed earlier.

While the growing of algae is well known, one of the difficulties in the industry is growing commercially useful amounts of algal biomass in a commercial time period. As the algal slurry increases in density, light transmissivity drops considerably with distance from the surface of the media. Thus for larger scale production, the depth of algal slurry through which light must travel seriously affects the growth rate of algae with algae further below the surface having a much slower growth rate than algae near the surface. Therefore to maximize grow, the algae must not be more than about 30 cm from the surface of the media. FIG. 14 shows a combined cultivation system used to produce algae at a large volume in a commercial time period. In this embodiment, the volume of each cultivation chamber is increased in each stage.

In the first stage, CO₂ containing gas 69 mixed with aqueous nutrient media in a mixing tank 70 or like device is supplied to vertical growth columns 60 to initially grow the algae to a sufficient concentration to be used as a feed to larger volume cultivation chambers. This ensures that sufficient algae culture is introduced into the small cultivation chambers 100 for sufficient biomass to be grown in an commercially acceptable time period. Cultivation chambers 100 are typically 10 m in length and of a TAG construction described earlier. Cultivation chambers 100 are sized and controlled so that the residence time in the small cultivation chambers 100 produces sufficient biomass to be used as a feed for the large cultivation chambers 200, 200a. The sizing and control will depend on the levels of sunlight expected and actually received at the location of the plant, the acceptable residence time in the chambers, typically 24 hours and the growth rate of the culture.

The algal slurry then passes to cultivation chamber 200 and then circulated through cultivation chambers 200a to maintain exposure of a sufficient volume of media and algae to light to enable photosynthesis to continue at an acceptable rate. These cultivation chambers are preferably 50 m in length. Once the algal slurry has reached an acceptable density, it then passes to a harvest system 71.

It would be appreciated by those skilled in the art that the sizing of the cultivation chambers may vary depending on the available footprint of land available. However in accordance with this embodiment, the sizing of the cultivation chambers is progressively larger than the chambers in the previous section.

It will be understood that the invention disclosed and defined in this specification extends to all alternative combinations of two or more of the individual features mentioned or evident from the text or drawings. All of these different combinations constitute various alternative aspects of the invention.

1. A biological cultivation system for the culture of photosynthetic organisms including
at least one cultivation chamber permitting exposure of the culture medium to natural and/or artificial light and including;
a light transmissive wall or walls defining a gas space; and
a culture medium containment area below the gas space;
one or more fluid inlets positioned within the culture medium containment area; and
one or more gas outlets in communication with the gas space;
a control unit operatively connected to a gas flow control device, the gas flow control device controlling the flow of gas in through the fluid inlets and out through the fluid outlets to control the conditions within the cultivation chamber.

2. The biological cultivation system of claim 1 further including a plurality of cultivation chambers interconnected in series or in parallel.

3. The biological cultivation system of claim 1 further including a plurality of cultivation chambers wherein the cultivation chambers include
one or more chambers formed of a flexible material; and
one or more chambers including a pair of opposed substantially rigid walls enclosed by a light transmissible section.

4. The biological cultivation system of claim 1 wherein the cultivation chambers includes one or more fluid ports to allow for the introduction and removal of the culture medium, the one or more fluid ports including a regulator to control the introduction and removal of the culture medium from the cultivation chamber.

5. The biological cultivation system 1 wherein the fluid inlets for the culture medium containment area are positioned along a base portion of the culture medium containment area.

6. The biological cultivation system of claim 5 wherein the cultivation chamber is in the form of an enclosed flexible plastic structure of tube-like configuration.

7. The biological cultivation system of claim 5 wherein the one or more walls of the cultivation chamber are light-transmissible and the walls are integrally formed in a tubular shape.

8. The biological cultivation system of claim 5 wherein the cultivation chamber is horizontally oriented producing a flat base.

9. The biological cultivation system of claim 8 wherein the base has a slope in the range of 1-5° towards the discharge end of the chamber.

10. The biological cultivation system of claim 6 wherein the one or more walls are formed from a material to permit transmission of light at a pre-determined wavelength, material permitting UV light transmission of approximately 20% to 65%.

11. The biological cultivation system of claim 1 wherein the cultivation chamber is inflatable, the inflation of the cultivation chamber being maintained by the flow of gas achieved through the introduction of gas into the chamber through the gas inlets and out through the gas outlets, the gas inlets being positioned above and/or below the surface of the culture medium.

12. The biological cultivation system of claim 1 further including a secondary light control device to control the amount of light transmitted through the walls of the cultivation chambers.

13. The biological cultivation system of claim 12 wherein the secondary light control device is fixed and/or variable and includes a shade sail or cloth.

14. The biological cultivation system of claim 5 wherein a plurality of fluid inlets are positioned along the length of the base of the cultivation chamber.

15. The biological cultivation system of claim 14 wherein the fluid inlets are positioned along conduits at the base of the cultivation chamber, wherein the conduits are adapted to carry and distribute the flow of gas, the gas inlets being positioned along the conduits at regular intervals to allow for a substantially even distribution of gas flow along the length of an cultivation chamber.

16. The biological cultivation system of claim 16 wherein the gas outlets include a a one-way valve system to allow gas to be released from the containment chamber.

17. The biological cultivation system of claim 1 further including:
(i) at least one vertically oriented growth column including
a light transmissible conduit;
one of more fluid inlets in communication with the conduit; and
one or more fluid outlets; and
wherein a fluid outlet of the growth column is flow-connected to a fluid inlet of the cultivation chamber.

18. The biological cultivation system of claim 1 wherein the light transmissive conduit includes a light transmissive inner conduit and a light transmissive outer conduit surrounding and in fluid communication with the inner conduit.

19. The biological cultivation system of claim 1, the control unit further including
a culture medium input system; with at least one cultivation chamber and/or at least one vertically oriented growth column being flow-connected to the control unit, the fluid inlets and outlets of the cultivation chambers and growth columns permitting controlled circulation of the culture medium.

20. The biological cultivation system of claim 19 wherein the control unit further includes a drive controller(s) to control pumping rates of the culture medium in combination with a programmable logic control (PLC).

21. The biological cultivation system of claim 20 wherein the drive controller functions to control both culture growth and pump control.

22. The biological cultivation system of claim 20 wherein the control unit includes a vector based combination drive and input/output (I/O) interface to combine pump and process control function.

23. A method of cultivating photosynthetic organisms may include:
(a) providing a cultivation chamber including:
(i) at least one light transmissive wall or walls defining:
a gas space; and
a culture medium containment area below the gas space;
(ii) one or more gas inlets and one or more gas outlets in communication with the gas space;
(iii) one or more fluid outlets and outlets communicating with the culture containment are; and
(iii) a control unit including a gas flow control device;
(b) introducing into the cultivation chamber a culture medium and an inoculate of photosynthetic organisms, the cultivation chamber permitting exposure of the culture medium to a natural and/or artificial light;
(c) controlling the flow of gas in through the gas inlets and out through the gas outlets using the gas flow control device, wherein the flow of gas drives evaporation from the culture medium and/or controls the temperature of the cultivation chamber; and
(d) allowing the photosynthetic organisms to grow in the presence of light.

24. The method of claim 23 wherein fluid inlets are positioned along a base portion of the culture medium containment area.

25. The method of claim 24 wherein the first gas includes carbon dioxide (CO₂) to provide carbon to the photosynthetic organisms and/or reduce the pH of the circulating fluid.

26. The method of claim 24 wherein gas is passed into the cultivation chamber through fluid inlets both above the surface of the culture medium. and below the surface of the culture medium, the introduction of the gas above the surface of the culture medium allowing for a modification of the atmosphere in the cultivation chamber.

27. The method of claim 23 further including the steps of
providing an effective amount of a selective biocide; and
treating the culture medium with the selective biocide to reduce or eliminate growth of unwanted organisms.

28. The method of claim 27 wherein the unwanted organism is a parasite, bacterium, fungal or algal strain and the biocide is selected from the group consisting of include a pesticide, bactericide, fungicide or algaecide or a combination thereof.

29. Original) The method of claim 28 wherein the biocide is copper sulphite.

30. The method of claim 23 wherein the gas flow control device functions to create a positive atmospheric displacement differential between the liquid culture medium and the gas space.

31. The method of claim 30 wherein the gas flow control device performs the steps of
(i) metering of gas delivery volume within the cultivation chamber on the delivery feed side and
(ii) releasing excess gas pressure from gas space above liquid medium via a pressure release mechanism.

32. The method of claim 23 wherein the control of the evaporation from the culture medium provides a degree of control over the temperature within the cultivation chamber.

33. The method of claim 34 wherein the gas flow control device further includes a temperature control.

34. The method of claim 32 the rate at which gas is introduced into the cultivation chamber and/or the temperature of the introduced gas is used to control the temperature of the culture medium or gas space in the cultivation chamber.

35. The method of claim 19 further includes the steps of reducing or eliminating the nutrient content of the culture medium for a pre-determined period; and harvesting the photosynthetic organisms.

36. A method of cultivating photosynthetic organisms of claim 23 further including the steps of:
(a) introducing into at least one vertical growth column, a culture medium and an inoculate of photosynthetic organisms; the at least one vertically oriented growth column including
a light transmissive conduit; and
a cultivation chamber including a wall or walls defining a gas space and a culture medium containment area below the gas space;
(b) introducing a first gas into the vertical growth column; and allowing the photosynthetic organisms to grow in the presence of light to form an algal slurry;
(c) introducing the algal slurry of step (b) into the cultivation chamber, the cultivation chamber permitting exposure to natural and/or artificial light;
(d) introducing a first gas through the inlet(s) within the containment area, wherein the flow of gas thereby mixes the culture medium;
(e) introducing a second gas through the gas inlet(s) into the gas space, wherein the second gas functions to control the temperature of the gas space; and
(f) allowing the photosynthetic organisms to grow further in the presence of light.

37. The method of claim 36 wherein the vertically oriented growth column includes a light transmissible inner conduit and a light transmissible outer conduit.

38. The method of claim 36 wherein gas passes into the base of the inner conduit via the gas inlets and bubbled into the culture medium.

39. The method of claim 36 wherein an array of vertical growth columns are used, the vertical growth columns in the array being the same or different and the columns are arranged in series or in parallel.

40. The method of claim 36 wherein the growth columns are arranged in series, culture medium and photosynthetic organisms which forms an algal slurry from one column become the feedstock for an adjacent column.

41. The method of claim 22 wherein the algal slurry is passed through the fluid outlet(s) to the cultivation chamber.

42. The method of claim 36 wherein gas is then be passed into the cultivation chamber via the fluid inlets situated in the cultivation containment area.

43. A method for the conversion of carbon dioxide to algal biomass including the steps of:
cultivating algal photosynthetic organisms by the method of claim 23 in the presence of light wherein the first gas is carbon dioxide.

44. A method of recycling emitted carbon dioxide by utilising the emitted carbon dioxide comprising the steps using the emitted carbon dioxide containing gas as an input in the production of photosynthetic organisms using the method of claim 23.

45. The method of claim 44 wherein the emitted carbon dioxide containing gas is cooled and partly scrubbed of pollutants before it is introduced into the cultivation chamber.

46. The method of claim 23 further including the steps of
(g) recovering the photosynthetic organisms from step (f) including the steps of:
providing photosynthetic organisms as a concentrated photosynthetic organism biomass having substantially intact cells;
(ii) homogenising the biomass using a mechanical homogeniser to disrupt the cells of the microorganisms; and
(iii) separating the homogenised biomass into fractions.

47. A photosynthetic growth system including a plurality of cultivation chambers of claim 1 arrange in two or more sections, wherein
the sections are connected in series and the cultivation chambers in the first section includes
at least one vertically oriented growth column including
a light transmissible conduit;
and the cultivation chambers in each section are either
of a greater volume/capacity than the cultivation chambers of a previous section in the series; or
the total volume/capacity of cultivation chambers in each section is greater than the previous section.
